(19) United States
(12) Patent Application Publication
(10) Pub. No.: US 2021/0023141 A1
(43) Pub. Date: Jan. 28, 2021

(54) STEM CELLS AND PROGENITOR CELLS BEARING CD31

(71) Applicants: SYDDANSK UNIVERSITET, Odense M (DK); REGION SYDDANMARK, Vejle (DK)

(72) Inventors: Søren Paludan SHEIKH, Klampenborg (DK); Ditte Caroline ANDERSEN, Odense SV (DK); Charlotte Harken JENSEN, Odense SV (DK)

(21) Appl. No.: 17/042,763

(22) PCT Filed: Apr. 3, 2019
(86) PCT No.: PCT/EP2019/058405

§ 371 Date: Sep. 28, 2020

(30) Foreign Application Priority Data
Apr. 4, 2018 (DK) PA 2018 70194

(51) Int. Cl. A61K 35/28; A61P 15/10; G01N 33/569; A61P 35/00; A61P 1/00

(52) U.S. Cl. CPC A61K 35/28; A61P 15/10; A61P 1/00; A61P 35/00; G01N 33/56966

(57) ABSTRACT

The present invention relates to an isolated CD31+ cell derived from adipose-derived regenerative cells (ADRC's), for use as a medicament. The invention also relates to compositions comprising such cells. In addition, the invention relates to methods for screening for the regenerative capacity of a population of adipose-derived regenerative cells (ADRC's).

### TECHNICAL FIELD OF THE INVENT ION

The present invention relates to specific isolated adipose-derived regenerative cells (ADRCs) also often referred to as adipose derived stromal vascular fraction (SVF). In particular, the present invention relates to medical uses of CD31+ ADRCs. In addition, the invention relates to methods for screening for the regenerative capacity of a population ADRCs.

### BACKGROUND OF THE INVENTION

Adipose-derived regenerative cells (ADRCs) are obtained through adipose tissue, most often as a lipoaspirate following liposuction. The ADRC is heterogeneous and contains multiple cell types, including mesenchymal stem cells that have been shown to originate from the perivascular space in vivo, and consequently possess strong regenerative capacity for vascular structures among others. They generate a regenerative microenvironment by secreting molecules including hormones, growth factors and cytokines (+non-coding small RNAs) that facilitate repair after injury; thus also working as site-regulated ‘drugstores’ in vivo and not only as stem cells with vascular potential. Their therapeutic capabilities have just recently begun to be understood, and they can readily differentiate along distinct lineage pathways and give rise to cells in bone, cartilage, muscle, blood vessels and other mesenchymal tissues. Furthermore, ADRCs are capable of trans-differentiation into ectodermal and endodermal lineages including endocrine pancreatic cells, hepatocytes and various neuronal cell types.

The mechanistic actions of the ADRC's both from a cellular transplant and a host perspective are not completely understood. It is debated whether transplanted ADRCs engraft to restore the damaged tissue with new cells or if they perform a transient beneficial role either by a direct ADRC-tissue cell interaction or indirectly by secreting regenerating molecules that modulates the tissue cells. This is a very important question, and seems to depend on the tissue in which the ADRCs are injected. It is also highly likely that all three scenarios appear simultaneously and synergistic from different types of cells in the heterogeneous ADRC population. ADRC's have a massive regenerative potential, as seen for e.g. erectile dysfunction and lymphedema following transplantation. For example Haahr et al. (EBioMedicine 5 (2016) 204-210) discloses intracavernous Injection of Autologous Adipose-Derived Regenerative Cells in Patients with Erectile Dysfunction Following Radical Prostatectomy. Haahr et al. is silent in respect of the cells being CD31 and/or CD34 positive. Toyserkani et al. (Stem Cells Translational Medicine 2016;5:857-859) discloses treatment of lymphedema using isolated adipose-derived stromal cells. Toyserkani et al. is silent in respect of the cells being CD31 and/or CD34 positive.

WO 2014/138793 discloses methods for isolating endothelial progenitor cells (EPCs). More particularly, methods for isolating endothelial progenitor cells that exhibit self-renewal and differentiation capacity is disclosed. It is further disclosed that the highest endothelial stem cell activity resides in the CD31⁻ cells, whereas CD31⁺ cells provide only limited colony forming potential.

Hence, an improved isolated population of ADRC's with regenerative properties would be advantageous, and in particular, a more efficient and/or reliable treatment protocol with such ADRC's would be advantageous.

### SUMMARY OF THE INVENTION

It is essential for the effects of ADRCs on e.g. erectile dysfunction and lymphedema that the injected stem cell suspension contains an optimal mixture of different cell types and the substances that may facilitate regenerative processes.

The ADRC solution contains a relatively heterogeneous cell population consisting of B and T lymphocytes, endothelial cells, fibroblasts, macrophages, pericytes, pre-adipocytes and other cells. In here it is disclosed that it is important that CD31 and CD34 positive cells are present in the ADRC suspension and in particular CD31 positive cells. These are two markers often used to distinguish vascular cells and stem cells from other cells, such as blood and stromal cells. The present inventors have used flow cytometric activated cell sorting to sort out CD31⁺/CD34⁺, CD31⁺/D34⁻, CD31⁻/CD34⁻, CD31⁻/CD34⁺ ADRCs. The number of injected ARDC's is likely also important for the effects on angiogenesis, lymphangiogenesis and the clinical efficiency.

Thus, an object of the present invention relates to the provision of isolated populations of ADRC's with regenerative properties. Importantly, example 4 shows in a phase 1 clinical trial that in a single intracavernous injection of autologous human ADRC's improves erection in men with erectile dysfunction (ED) after radical prostatectomy. Further, increasing the amount of CD31+ cells in the ADRC's improves the treatment.

Example 5 shows in a phase 1 clinical trial that in a single injection of autologous ADRC's in women with Breast Cancer Related Lymphedema (BCRL). Further, increasing the amount of CD31⁺ cells in the ADRC's improves the treatment.

Example 7 shows that a CD31⁺ human ADRC subpopulation stimulates tube formation to a higher extent than the CD31⁻ counterpart. Notably, the CD31⁺ ADRCs possesses superior angiogenic properties compared to the unfractionated 15 ADRCs.

In addition, example 8 shows in a phase 1 clinical trial that freshly isolated autologous adipose derived regenerative cells effectively heal Crohn's disease associated fistulas. Again, there seems to be a correlation of the highest healing efficacy in the patients receiving high amounts of CD31 expressing cells.

An aspect of the present invention relates to a composition comprising a population of isolated (viable) CD31⁺ cells derived from adipose-derived regenerative cells (ADRC), for use as a medicament. Preferably, the isolated (viable) CD31⁺ cells derived from adipose-derived regenerative cells (ADRC) constitutes at least 80% of the adipose-derived regenerative cells (ADRC) cells in the composition, such as at least 90%, such as at least 95%, such as at least 99% or such as 100%.

Another aspect of the invention relates to an isolated CD31⁺ cell derived from adipose-derived regenerative cells (ADRC), for use as a medicament.

Yet another aspect of the present invention relates to a kit comprising
i. the isolated cell according to the invention, and/or the composition according to the invention; and
ii. instructions for use for the treatment and/or alleviation of a disorder.

Preferably the kit comprises the composition according to the invention.

Still another aspect of the present invention is to provide a method for screening for the regenerative capacity of a population of adipose-derived regenerative cells (ADRC) from a subject, the method comprising
providing a population of isolated adipose-derived regenerative cells (ADRCs) from a subject,
determining the number of (viable) cells in the population being CD31+;
comparing said number to a first reference levels;
determining that
   said population of adipose-derived regenerative cells (ADRC) is indicative of a high regenerative capacity, if said number of CD31+ cells is above the first reference level, or
   said population of adipose-derived regenerative cells (ADRC) is indicative of a low regenerative capacity, if said number of CD31+ cells is equal to or below said first reference level.

As previously explained, example 4, 5 and 8 show (in phase 1 clinical trials) that ADRC's with a high number of CD31+ cells are more efficient as medicaments in relation to regenerative capacity. This is also confirmed in example 7.

### BRIEF DESCRIPTION OF THE FIGURES

FIG. 1 shows phenotypes of ASCs (cultured ADRC/SVF cells) and ADRC/SVF cells. The levels of mesenchymal stem cell markers of ASCs (black) and ADRC/SVF (white) cells were assessed by flow cytometry and found to be significantly different between the two groups of cells (****p<0.0001). Data is shown as mean ± SD, n=3 and statistical significance was tested using a two-way ANOVA test and Bonferroni post test. ***p<0.001, ****p<0.0001.

FIG. 2 shows that human induced Pluripotent Stem Cells (hiPSC) co-express CD31 and CD34 during vascular differentiation. Following mesoderm specification and further vascular differentiation, a fraction of iPS cells begin to express CD31 and CD34 simultaneously. Flow cytometry reveals that most, if not all vascular progenitors co-express the two membrane markers (2A). In each quadrant the population percentage is shown (mean ± SD, n=3). Populations were gated using FMO controls (2B and 2C).

FIG. 3 shows the effect of injecting different populations of ADRC's in subjects suffering from erectile dysfunction. Y-axis—Top: Erectile function scored according to the IIEF5 score. Y-axis—Bottom: Erectile function scored according to the EHS score. X-axis: A: Number of injected ADRC's. B: Number of injected CD31+ cells. C: Number of injected CD34+ cells. D: Percentage of injected CD31+ cells in the ADRC population. E: Percentage of injected CD34+ cells in the ADRC population. The data shows that injection of increasing numbers from 0 to 4.2×10⁶ of CD31 (CD31+) expressing ADRC's strongly correlated with recovery of erectile function.

FIG. 4 shows the 6 month data from 9 Lymphedema patients in relation to the effect of injecting different populations of ADRC's in patients suffering from lymphedema. Data is expressed as percentage reduction in scores from 0 to 6 months (((score, t=0−score, t=6 mo)/score, t=0)×100)). The higher the better. Y-axis—Top: scored according to the DASH score. Y-axis—Middle: scored according to heaviness score. Y-axis—Bottom: scored according to tension score. X-axis: A: Number of injected ADRC's. B: Percentage of injected CD31+ cells. C: Number of injected CD31+ cells. D: Percentage of injected CD235a-CD45-CD31+CD34+ cells. E: Number of injected CD235a-CD45-CD31+CD34+ cells.

FIG. 5 shows the 12 month data from 9 Lymphedema patients in relation to the effect of injecting different populations of ADRC's in patients suffering from lymphedema. Data is expressed as percentage reduction in scores from 0 to 6 months (((score, t=0−score, t=6 mo)/score, t=0)×100)). The higher the better. Y-axis—Top: scored according to the DASH score. Y-axis—Middle: scored according to heaviness score. Y-axis—Bottom: scored according to tension score. X-axias: A: Number of injected ADRC's. B: Percentage of injected CD31+ cells. C: Number of injected CD31+ cells. D: Percentage of injected CD235a-CD45-CD31+CD34+ cells. E: Number of injected CD235a-CD45-CD31+CD34+ cells.

FIG. 6 shows identification of CD31 + positive cells in the heterogeneous ADRC suspension by flow cytometry and APC conjugated antibodies. An APC conjugated isotype control was used to mark the entire CD31 negative population (left scatter plot) leaving less than 1% in the CD31+ gate. In this figure the APC-CD31 stained sample (right) showed that 20.55% (21.4−0.85) of the ADRCs were CD31+.

FIG. 7 shows that ADRC-derived CD31+ cells facilitate tube formation ex vivo. A-E) Phase contrast microscopic images of tubes sprouting from corpus cavernous tissue explant. A) +VEGF. B) normal growth medium. C) Isolated ADRC's. D) CD31⁺ ADRC cells. E) CD31⁻ ADRC cells. F) Tube counts. Values represent mean + SEM of n=4 independent experiments; One-way ANOVA followed by a Tukeys multiple comparisons test was used to test the difference between the CD31-positive and CD31-negative group; *p=0.0126.

The present invention will now be described in more detail in the following.

### DETAILED DESCRIPTION OF THE INVENTION

Definitions

Prior to discussing the present invention in further details, the following terms and conventions will first be defined:

CD31

Platelet endothelial cell adhesion molecule (PECAM-1) also known as CD31 is a protein that in humans is encoded by the PECAM1 gene. CD31 is found on the surface of platelets, monocytes, neutrophils, and some types of T-cells, and makes up a large portion of endothelial cell intercellular junctions. The encoded protein is a member of the immunoglobulin superfamily and is likely involved in leukocyte migration, angiogenesis, and integrin activation. CD31 is normally found on endothelial cells, platelets, macrophages and Kupffer cells, granulocytes, T/NK cells, lymphocytes, megakaryocytes, osteoclasts and neutrophils.

CD34

CD34 is a cell surface glycoprotein and functions as a cell-cell adhesion factor. It may also mediate the attachment of stem cells to bone marrow extracellular matrix or directly to stromal cells. CD34 is also the name for the human gene that encodes the protein. The CD34 protein is a member of a family of single-pass transmembrane sialomucin proteins that show expression on early hematopoietic and vascular-associated tissue. Cells expressing CD34 are normally found in the umbilical cord and bone marrow as hematopoietic cells, a subset of mesenchymal stem cells, endothelial progenitor cells, endothelial cells of blood vessels but not lymphatics (except pleural lymphatics), mast cells, a sub-population dendritic cells (which are factor XIIIa negative) in the interstitium and around the adnexa of dermis of skin, as well as cells in soft tissue tumors like DFSP, GIST, SFT, HPC.

Expression Level

In the present context (exemplified by CD31), the term “CD31+” or “CD31⁺” or “CD31 positive” is to be understood as cells expressing CD31 at any level above background level. Thus, the term refers to the expression level of CD31, in that the expression level of this cell surface marker is above background by comparison with the expression level of that marker in the population of cells being analyzed as a whole.

The terms “+”and “−” in relation to expression levels are known in the art and refer to the expression level of the cell marker of interest, in that the expression level of the cell marker corresponding to “+”is high, intermediate or low and the expression level of the cell marker corresponding to “−” is null or at least no higher than the background level.

Adipose-Derived Regenerative Cells (ADRC)

Adipose-derived regenerative cells (ADRCs), also referred to as stromal vascular fraction (SVF) are able to differentiate into vascular cells and neurons in vitro and a large body of preclinical work shows a surprisingly good effect of ADRC injection into the corpora cavernosa.

Lymphedema

Lymphedema, also known as “lymphoedema” and “lymphatic edema”, is a condition of localized fluid retention and tissue swelling caused by a compromised lymphatic system, which normally returns interstitial fluid to the thoracic duct, then the bloodstream. The condition can be inherited or can be caused by a birth defect, though it is frequently caused by cancer treatments including surgery and radiation, such as treatment of breast cancer.

Morbus Crohn

Morbus Crohn (or Crohn's disease) is a type of inflammatory bowel disease (IBD) that may affect any part of the gastrointestinal tract from mouth to anus.

Flap Surgery

Flap surgery is a technique in plastic and reconstructive surgery where any type of tissue is lifted from a donor site and moved to a recipient site with an intact blood supply. This is similar to but different from a graft, which does not have an intact blood supply and therefore relies on growth of new blood vessels.

Erectile Dysfunction

Erectile dysfunction (ED), also known as impotence, is a type of sexual dysfunction characterized by the inability to develop or maintain an erection of the penis during sexual activity. Prostate cancer is the most common cancer in men, and radical prostatectomy (RP) often results in erectile dysfunction (ED) and a substantially reduced quality of life. Other causes are cardiovascular disease and diabetes, neurological problems (for example, trauma from prostatectomy surgery), hormonal insufficiencies (hypogonadism), lower urinary tract infections and drug side effects. In addition, age is an independent risk factor for ED.

IIEF-5 Scoring

The IIEF-5 score (The International Index of Erectile Function) is the sum of the ordinal responses to different defined items.
22-25: No erectile dysfunction
17-21: Mild erectile dysfunction
12-16: Mild to moderate erectile dysfunction
8-11: Moderate erectile dysfunction
5-7: Severe erectile dysfunction

Thus, in the present context, the term “treatment of erectile dysfunction” may refer to improving an IIEF-5 scoring to 22-25, and the term “alleviation of erectile dysfunction” may refer to improving an IIEF-5 scoring by one or more point or improving the IIEF-5 scoring to a different category.

The Erection Hardness Score (EHS)

The Erection Hardness Score (EHS) can be a helpful tool to evaluate erectile dysfunction (ED)—a man's inability to get or maintain an erection firm enough for 20 sex. Developed in 1998, the EHS is a single-item Likert scale that men can use on their own. The tool asks them to consider the question “How would you rate the hardness of your erection?” and select one of the following options:
0—Penis does not enlarge.
1—Penis is larger, but not hard.
2—Penis is hard, but not hard enough for penetration.
3—Penis is hard enough for penetration, but not completely hard.
4—Penis is completely hard and fully rigid.

Thus, in the present context, the term “treatment of erectile dysfunction” may refer to improving an EHS scoring to 4, and the term “alleviation of erectile dysfunction” may refer to improving EHS scoring by one or more point.

Medical Uses of CD31⁺ Cells Derived from Adipose-Derived Regenerative Cells

As described in the example section, in here it has been found that a cell population of CD31⁺ cells derived from adipose-derived regenerative cells (ADRC) are important for the regenerative potential of ADRC's. Thus, an aspect of the invention relates to an isolated CD31⁺ cell derived from adipose-derived regenerative cells (ADRC), for use as a medicament.

It is known to use ADRC's for the treatment and/or alleviation of a medical disorder. Thus, in an embodiment, the isolated CD31⁺ cell is for use in the treatment and/or alleviation of a disorder selected from the group consisting of erectile dysfunction (ED), lymphedema, Morbus Crohn's disease, type 1 diabetes, type 2 diabetes, for promoting angiogenesis in cardiovascular including revascularization and regeneration of the heart and aorta aneurisms, regeneration of skin wounds, bone and cartilage disorders, fertility disorders, rheumatic diseases scleroderma, muscular diseases including sphincter regeneration, gastrointestinal repair, and diseases of the central and peripheral nervous system, preferably for the treatment and or alleviation of erectile dysfunction (ED), Morbus Crohn's disease or lymphedema.

In another embodiment, the isolated CD31⁺ cell is for use in the treatment and/or alleviation of a disorder selected from the group consisting of breast augmentation, general scar, burn and wounds, facial rejuvenation, reconstruction, androgenic alopecia, vulvar lichen sclerosus, peyronie's disease, urinary incontinence, faecal incontinence, anal fistula, multiple sclerosis, critical limb ischaemia, diabetic foot ulcer and osteoarthritis. These major applications of SVF-and ADSC-based therapeutics are also reviewed in Bora and Majumdar (Stem Cell Research & Therapy (2017) 8:145).

In another embodiment, the erectile dysfunction is erectile dysfunction following radical prostatectomy (RP), such as erectile dysfunction in subjects considered to suffer from nerve injury after radical prostatectomy (RP) or subjects who are incontinent. Example 4 shows data relating to treatment of erectile dysfunction following radical prostatectomy (RP). Further, the common understanding of ED after prostate surgery is due to damage to blood vessels but may also be due peripheral nerve damage. Thus, in yet an embodiment, the CD31+ cells according to the invention are for use in regeneration of nerves, preferably peripheral nerves. Damage to nerves may also cause phantom pains. Thus, in another embodiment, the CD31+ cells according to the invention are for use in the treatment or alleviation of phantom pain.

In a further embodiment, the isolated CD31⁺ cell is for use in the treatment and/or alleviation of Morbus Crohn's disease, an in particular treatment of fistulas associated with Morbus Crohn's disease. Example 8 shows such treatment of Morbus Crohn's disease associated fistulas.

In yet an embodiment, the isolated CD31+ cells are for improving vascular differentiation. In yet another embodiment, the isolated CD31+ cells are for improving angiogenesis.

In yet a further embodiment, the isolated CD31+ cells are used for facilitating tissue regeneration/repair/remodelling by other means than by directly involving vasculo-/angiogenesis, e.g. by modulation of inflammation or resolving fibrosis.

An advantage of using ADRC's are that they may be used very fast after having been provided from a subject. Thus, in yet an embodiment, the cell has been obtained from a subject within 24 hours before said use, such as within 12 hours, such as within 6 hours, such as within 3 hours before said use. In a further embodiment, said cell is a non-expanded cell.

Since an efficient subpopulation has been identified, the use of a similar subpopulation of expanded cells may also be foreseen. Thus, in an embodiment, 25 wherein said cell is an expanded cell.

It may be an advantage that the cells are used in the same subject from which they have previously been obtained. Thus, in a further embodiment the CD31⁺ cell is for use as an autograft.

The cells may however also find use in other subjects. Thus, in an embodiment the CD31⁺ cell (or composition comprising the CD31+ cells) is for use as an allograft or isograft.

In yet a further embodiment, the isolated CD31⁺ cell is for use in the treatment or alleviation of a mammal, preferably a human.

In an additional embodiment, said cell is also CD34⁺. In another embodiment, said cell is CD235a⁻ and/or CD45⁻.

Composition Comprising CD31⁺ Cells Derived from Adipose-Derived Regenerative Cells (ADRC's)

The CD31+ cells (or cell population) may be comprised in a population of other cells or cell types. Thus, another aspect of the invention relates to a composition comprising a population of cells according to the invention for use as a medicament.

In an embodiment, the composition is for use in the treatment or alleviation of a disorder selected from the group consisting of erectile dysfunction (ED), lymphedema, Morbus Crohn's disease, type 1 diabetes, type 2 diabetes, for promoting angiogenesis in cardiovascular including revascularization and regeneration of the heart and aorta aneurisms, regeneration of skin wounds, bone and cartilage disorders, fertility disorders, rheumatic diseases scleroderma, muscular diseases including sphincter regeneration, gastrointestinal repair, and diseases of the central and peripheral nervous system, preferably for the treatment and/or alleviation of erectile dysfunction (ED), Morbus Crohn's disease or lymphedema.

In another embodiment, the erectile dysfunction is erectile dysfunction following radical prostatectomy (RP). In a related embodiment, said subject is not incontinent. Thus, in a preferred embodiment, the composition is for use in the treatment and or alleviation of erectile dysfunction. Example 4 shows that the amount of CD31+ cells are important in the treatment of ED.

In yet an embodiment, the composition is for use in the treatment and or alleviation of lymphedema, preferably breast cancer related lymphedema. Example 5 shows that the amount of CD31+ cells are important in the treatment of breast cancer related lymphedema.

In a further embodiment, the composition is for use in the treatment and/or alleviation of Morbus Crohn's disease. Example 8 shows such treatment of Morbus Crohn's disease related fistulas. Further, the data in example 8 indicate that ADRC having high amounts of CD31+ cells give rise to the best treatment. In yet an embodiment, the composition is for use in the treatment and/or alleviation of Crohn's complicated by perianal fistulas. In yet an embodiment, the composition is for use in the treatment and/or alleviation of fistulas, such as fistulas resulting from Morbus Crohn's. In yet a further embodiment, the ADRC's is administered/injected on and/or around the fistula, e.g. together with an amount of adipose tissue.

The amount of CD31+ cells in the composition may vary. Thus, in an embodiment the amount of cells according to the invention (CD31+) constitutes at least 5% of the adipose-derived regenerative cells (ADRC) cells in the composition, such as at least 8%, such as at least 10%, such as at least 20%, such as at least 30%, such as at least 50%, such as at least 70%, such as at least 80%, such as at least 90%, such as at least 95%, or such as at least 99% of the viable adipose-derived regenerative cells (ADRC) cells in the composition or wherein the amount of CD31⁺ cells constitutes in the range 8-80%, such as in the range 8-50%, or such as 8-30%.

In a preferred embodiment the amount of cells (CD31+) according to the invention constitutes at least 80% of the adipose-derived regenerative cells (ADRC) cells in the composition, such as at least 90%, more preferably such as at least 95%, and even more preferably such as at least 99% or such as 100%. As shown in examples 4, 5 and 8, an increasing number of CD31+ cells improves treatment with ADRC's. Further, as shown in example 7, a subpopulation of ADRC's selected positively for CD31 are more efficient than the whole population and significantly more efficient than the population negatively selected for CD31.

In yet an embodiment, the amount of cells being CD34+ constitutes at least 30% of the adipose-derived regenerative cells (ADRC) cells in the composition, such as at least 40%, preferably at least 50%, such as at least 60%, such as at least 70% of the viable adipose-derived regenerative cells (ADRC) cells in the composition or wherein the amount of CD34⁺ cells constitutes in the range 50-90%, such as in the range 50-80%, or such as 60-80%. In another embodiment, at least 1×10⁶ of cells being CD34+ are administered to the subject, such as at least 5×10⁶ of cells, such as at least 6×10⁶ of cells, such as at least 1×10⁷ of cells.

Without being bound by theory, it is noted that it appears as if most cells being CD31+ are also CD34+, whereas not all cells being CD34+ are also CD31 positive.

In yet an embodiment, the composition is in the form of a pharmaceutical composition.

In a further embodiment, the composition is for use for administration by injection to the corpora cavernosa (intracavernous injection), with the proviso that it is for the treatment and/or alleviation of erectile dysfunction.

In yet a further embodiment, the composition for use for administration by injection to the subcutaneous compartment (e.g. in the armpit and upper arm region), with the proviso that it is for treatment and/or alleviation of (breast cancer related) lymphedema.

In a further embodiment, the composition is for use for administration by injection to the on and/or around a fistula, e.g. together with an amount of adipose tissue, with the proviso that it is for the treatment and/or alleviation of Morbus Crohn's disease (associated fistulas).

It has been found that the amount of CD31+ cells in the composition is important for achieving a successful treatment (see e.g. examples 4, 5, 7 and 8). Thus, in an embodiment at least 1.5×10⁶ of cells according to the invention are administered to the subject, such as at least 2×10⁶ of cells, such as at least 3×10⁶ of cells, such as at least 4×10⁶ of cells, such as at least 4.5×10⁶ of cells according to any of claims 1-9. In another embodiment, 3-15 million CD31+ ADRCs are administered, such as 5-15 million, or such as 7.75 to 12.3 million CD31+ ADRCs.

It has also been found that there appears to be an upper limit for the amount of CD31+ cells in the composition for the treatment to be effective (see example 4). Thus, in a further embodiment, in the range 2×10⁶ to 5×10⁶ CD31+ cells according to the invention are administered to the subject, such as in the range 2.5×10⁶ to 4.5×10⁶ are administered to the subject. The data in example 7 however indicates that cells selected positively for CD31 expression are very effective.

Kit of Parts

The cells and/or composition according to the invention may also form part of a kit or kit of parts. Thus, an aspect of the invention relates to a kit comprising
i. the isolated cell according to the invention, and/or the composition according to the invention; and
ii. instructions for use for the treatment and/or alleviation of a disorder, such as selected from the group consisting of erectile dysfunction (ED), lymphedema, Morbus Crohn's disease, type 1 diabetes, type 2 diabetes, for promoting angiogenesis in cardiovascular including revascularization and regeneration of the heart and aorta aneurisms, regeneration of skin wounds, bone and cartilage disorders, fertility disorders, rheumatic diseases scleroderma, muscular diseases including sphincter regeneration, gastrointestinal repair, and diseases of the central and peripheral nervous system, preferably for the treatment and or alleviation of erectile dysfunction (ED), Morbus Crohn's disease or lymphedema.

Process for Preparing an Isolated Cell Population

The present invention also relates to a process for preparing an isolated cell population. Thus, an aspect relates to a process for preparing an isolated cell population of viable ADRC cells, the process comprising
providing a (previously obtained) first population of freshly collected adipose-derived regenerative cells (ADRC); and
   I. providing a first subpopulation of adipose-derived regenerative cells (ADRC) from said first population by positively selecting for CD34+ cells; and
   II. providing a second subpopulation of adipose-derived regenerative cells (ADRC) from said first subpopulation by positively selecting for CD31+ cells; thereby providing a CD31+/CD34+ subpopulation of viable cells;
      OR
   I. providing a first subpopulation of adipose-derived regenerative cells (ADRC) from said first population by positively selecting for CD31+ cells;
   II. providing a second subpopulation of adipose-derived regenerative cells (ADRC) from said second subpopulation by positively selecting for CD34+ cells; thereby providing a CD31+/CD34+ subpopulation of viable cells;
      OR
   I. providing a first subpopulation of adipose-derived regenerative cells (ADRC) from said first population by positively selecting for CD31+/CD34+ cells; thereby providing a CD31+/CD34+ subpopulation of viable cells; p3 OR
   I. providing a first subpopulation of adipose-derived regenerative cells (ADRC) from said first population by positively selecting for CD31+ cells; thereby providing a CD31+ subpopulation of viable cells.

In a preferred embodiment, the process comprises providing a first subpopulation of adipose-derived regenerative cells (ADRC) from said first population by positively selecting for CD31+ cells; thereby providing a CD31+ subpopulation of viable cells.

In an embodiment, said selection of subpopulation is performed by cell sorting such as FACS.

In a related aspect, the invention relates to an isolated viable adipose-derived regenerative cell (ADRC), derived from adipose-derived regenerative cells (ADRC) adipose tissue, obtained/obtainable by a process according to the invention for use as a medicament.

In an embodiment, the isolated viable adipose-derived regenerative cell (ADRC), obtained/obtainable by a process according to the invention, is for use in the treatment and or alleviation of a disorder selected from the group consisting of erectile dysfunction (ED), lymphedema, Morbus Crohn's disease, type 1 diabetes, type 2 diabetes, for promoting angiogenesis in cardiovascular including revascularization and regeneration of the heart and aorta aneurisms, regeneration of skin wounds, bone and cartilage disorders, fertility disorders, rheumatic diseases scleroderma, muscular diseases including sphincter regeneration, gastrointestinal repair, and diseases of the central and peripheral nervous system, preferably for the treatment and or alleviation of erectile dysfunction (ED), Morbus Crohn's disease or lymphedema.

Method for Screening for the Regenerative Capacity of a Population of Adipose-Derived Regenerative Cells (ADRC)

It has been found that the amount of CD31+ cells in the ADRC population is important for the regenerative capacity of the cell population. Thus, yet a further aspect of the invention relates to a method for screening for the regenerative capacity of a population of adipose-derived regenerative cells (ADRC) from a subject, the method comprising
providing a (having provided a previously obtained) population of isolated adipose-derived regenerative cells (ADRC) from a subject,
determining the number of (viable) cells in the population being CD31+;
comparing said number to a first reference levels;
determining that
   said population of adipose-derived regenerative cells (ADRC) is indicative of a high regenerative capacity, if said number of CD31+ cells is above the first reference level, or
   said population of adipose-derived regenerative cells (ADRC) is indicative of a low regenerative capacity, if said number of CD31+ cells is equal to or below said first reference level.

In an embodiment, the method further comprising comparing said number of (viable) cells in the population being CD31+ to a second reference levels;
determining that
   said population of adipose-derived regenerative cells (ADRC) has a high regenerative capacity, if said number of CD31+ cells is below the second reference level, or
   determining that said population of adipose-derived regenerative cells (ADRC) has a low regenerative capacity, if said number of CD31+ cells is equal to or above said second reference level, with the proviso that the second reference level is higher than the first reference level.

If the number of CD31+ cells in the population fall outside the first and/or second reference level, it may be possible to adjust the level. Thus, in an embodiment, the method further comprises, if said number of CD31+ cells is below said first reference level, adjusting said level to a higher number/concentration. In a similar embodiment, the method further comprises, if said number of CD31+ cells is equal to or above said second reference level, adjusting or diluting said level to a lower number/concentration. However, example 7 indicates that a high number of CD31+ cells are preffered.

The percentage of CD31+ cells in the cell population may also be important. Thus, in a further embodiment, the method further comprises determining the percentage of viable CD31+ cells in the total population of (viable) isolated adipose-derived regenerative cells (ADRC);
comparing said number to a third reference level;
determining that
   said population of adipose-derived regenerative cells (ADRC) is indicative of a high regenerative capacity, if said percentage of CD31+ cells is above the third reference level, or
   said population of adipose-derived regenerative cells (ADRC) is indicative of a low regenerative capacity, if said percentage of CD31+ cells is equal to or below said third reference level.

In yet an embodiment, the method further comprising
comparing said percentage of (viable) cells in the population being CD31+ to a fourth reference level;
determining that
   said population of adipose-derived regenerative cells (ADRC) is indicative of a high regenerative capacity, if said percentage of CD31+ cells is below the fourth reference level, or
   said population of adipose-derived regenerative cells (ADRC) is indicative of a low regenerative capacity, if said percentage of CD31+ cells is equal to or above said second reference level, with the proviso that the fourth reference level is higher than the first reference level.

If the percentage of CD31+ cells in the population fall outside the third and/or fourth reference level, it may be possible to adjust the percentage. Thus, in an embodiment, the method further comprises, if said percentage of CD31+ cells is below said third reference level, adjusting said percentage to a higher percentage. In a similar embodiment, the method further comprises, if said percentage of CD31+ cells is equal to or above said fourth reference level, adjusting or diluting said level to a lower percentage.

Method of Treating a Medical Condition

In a further aspect, the invention relates to a method of treating a medical condition in a subject in need thereof, comprising administering to the subject a (pharmaceutical) composition according to the invention, in an amount effective to treat or alleviate the medical condition in the subject.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1

### Freshly Isolated But Not Cultured Adipose Derived Regenerative Cells Express CD31 and CD34

Aim

Adipose tissue can give raise to two stem cell populations: the freshly isolated stromal vascular fraction (SVF)/Adipose derived regenerative (ADRC cells and its cultured counterpart, the adipose tissue derived stromal/stem cells (ASCs). Both SVF/ADRC and ASCs exhibit regenerative effects and are suitable for clinical application, but it will be important to further characterize their differences including cellular makers and regenerative potential.

Materials and Methods

Flow Cytometry

The presence of different cellular markers in the two populations was characterized. Isolated SVF/ADRC cells or harvested ASCs (passage 4) were washed in HBSS/1% PS/5% FBS and fixed in HBSS/5% FBS/1% PS/1% NBF over night at 4° C. The cells were then washed twice and stored in HBSS/1% PS/5% FBS/0.05% Sodium-Azide at 4° C. until analysis. Fixed cells were washed in HBSS/1% PS/5% FBS and incubated 60 min with primary antibodies on ice while shaking. Following two washes, the cells were incubated 30 min with secondary antibodies on ice while shaking and finally washed twice.

Data Acquisition and Analysis

Data acquisition and analysis were obtained using a FACSCalibur instrument (Becton Dickinson, CA, USA) and Flowjo 10.0.6 software (Tree Star Inc, OR, USA), respectively. Primary antibodies were specific for rat CD45, CD90, CD44 (BD bioscience, 554875 (1:100), 554895 (1:50); and 554869 (1:100), respectively), CD29 (abcam, ab52971 (1:100)), CD34 (R&D systems, MN, USA, AF6518 (1:72)), PDGFRα (cell signaling, 3164 (1:200) and CD31 (Santa Cruz, sc-1506 (1:100)). Isotypes included sheep IgG (R&D systems, 5-001-A), rabbit polyclonal IgG, rabbit monoclonal IgG (abcam, ab37415, ab125938), mouse IgG2a,k, and mouse IgG1,k (Sigma-Aldrich, M 5409, M 5284). Alexa 488 or 647 conjugated secondary donkey antibodies specific for rabbit IgG, mouse IgG, sheep IgG (all purchased at Invitrogen, 1:200) were used for visualization.

Results

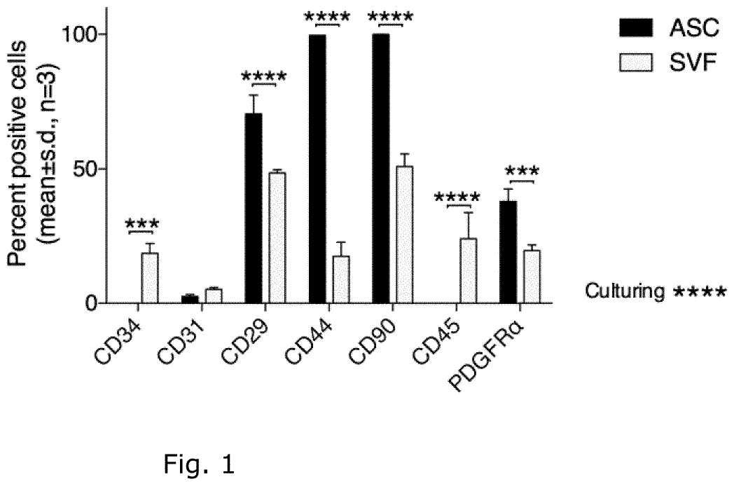

The immunophenotype of the two cell population groups was examined by flow cytometry analysis of several mesenchymal stem cell markers (FIG. 1). ADRC/SVF cells showed an overall phenotype of CD34⁺/CD31⁻/CD29⁺/CD44⁺/CD90⁺/CD45⁺/PDGFRα⁺, whereas the ASC groups showed overall phenotypes of CD34⁻/CD31⁻/CD29⁺/CD44⁺/CD90⁺/CD45⁻/PDGRF α⁺. These results concur with previously described ASC and ADRC/SVF marker profiles. The ADRC/SVF and ASC groups differed significantly in the levels of examined markers (ANOVA, p<0.0001, FIG. 1). In particular, levels of CD34 (p=0.0002) and CD45 (p<0.0001) were significantly higher in the ADRC/SVF population as compared to ASCs. The CD31 level was also higher in the ADRC/SVF population although the levels did not reach statistical significance in this study (FIG. 1 and table I). In contrast, levels of CD29 (p<0.0001), CD44 (p<0.0001), CD90 (p<0.0001) and PDGFRα (p=0.0003) were higher in the ASC compared to the SVF population.

TABLE I
Mesenchymal stem cell marker levels:
Comparison of SVF and ASC

| Marker | SVF | ASC | Significant difference |
|---|---|---|---|
| CD45 | 24 ± 9.69% | 0 ± 0% | p < 0.0001 |
| CD34 | 18.56 ± 3.68% | 0 ± 0% | p = 0.0002 |
| CD90 | 50.88 ± 4.74% | 99.93 ± 0.06% | p < 0.0001 |
| CD29 | 48.51 ± 1.20% | 70.4 ± 7.04% | p < 0.0001 |
| CD44 | 17.45 ± 5.23% | 99.56 ± 0% | p < 0.0001 |
| PDGFRα | 19.53 ± 2.19% | 37.82 ± 4.77% | p = 0.0003 |
| CD31 | 5.16 ± 0.75% | 2.52 ± 0.69% | Ns |

Ns: not significant

Conclusion

In summary, the freshly isolated cells (ADRC/SVF) exhibit a higher CD31 and CD34 expression than cultured stem cells (ASC).

### Example 2

### Human Freshly Isolated Adipose-Derived Stromal Cells (ADRC/SVF) Show Higher Flap Survival in a Rodent Model Than Cultured Adipose-Derived Stem Cells (ASC)

In plastic surgery, flaps are often used to cover defects where other reconstructive possibilities are insufficient. A common complication of flap surgery is insufficient blood supply leading to ischemia and tissue necrosis. The use of flaps is limited by their innate blood supply, and it has been hypothesized that stem cells can promote neoangiogenesis in the flap leading to increased flap survival. The ischemic flap in rodents has been widely used for stem cell research, and is generally accepted as one of the best models for studying the efficacy of stem cells to promote angiogenesis acutely in an in-vivo setting. This model is relevant for flap surgery as it can be viewed as an in-vivo assay of angiogenesis. By extension, the implications of the model for regenerative medicine are much broader as it can be regarded as a model for neoangiogenesis. In this model there is a clear contrast between surviving and necrotic skin since, the necrotic skin appeared dark and rigid whereas the surviving skin had a normal texture.

Results

The mean survival rates ± SD were 55.0 ± 7.2%, 50.4 ± 9.1% and 45.7 ± 9.5% for the ADRC/SVF, ASC and control group respectively. The ADRC/SVF improved flap survival better than ASC although the difference did not reach statistical significance. The difference between the ADRC/SVF and control group was statistically significant (p<0.05).

The vessel density was assessed by immunohistochemistry. Sections from each rat was stained for CD31 and alphaSMA to determine vessel density of flaps. The staining by aSMA showed no difference between any of the groups. The CD31 staining was increased in only the hSVF/ADRC (13.67 ± 2.54, p<0.01) as compared to the control group (10.32 ± 1.39). The lysate group was in between and not significant (11.75 ± 2.47).

Discussion

Very few studies have directly compared the efficacy of SVF/ADRC and ASC in in-vivo studies. Harada et al compared the efficacy of SVF and ASCs in immunocompromised mice in a hindlimb ischemia model where they found similar results as ours where the SVF/ADRC resulted in increased blood flow in the limb. In addition, they performed an in-vitro angiogenesis assay where the SVF was shown to be superior at promoting endothelial cell sprouting (Biomed Res. (2013) 34:23-9). In an entirely different kind of model, Semon et al showed that the SVF/ADRC was superior to ASCs in ameliorating experimental autoimmune encephalitis in immunocompetent mice (Stem Cells Transl Med. (2013) 2:789-96). These findings have not been confirmed by Bai et al when used for myocardial ischemia where the SVF/ADRC and ASCs performed equally well ((Eur Heart J (2010) 31:489-501).

Conclusion

Since the SVF/ADRC improved flap survival better than ASC and the SVF/ADRC have increased expression of CD31 and CD34, it is suggested that these two molecular markers play a role in mediating the increased biological efficiency of the SVF/ADRC stem cells.

### Example 3

### Human Induced Pluripotent Stem Cells (iPS cells) Express CD31 and CD34 When They Differentiate into Vascular Smooth Muscle Cells

Materials and Methods

We examined the early differentiation stages of human induced pluripotent stem cells (hiPSCs) into mesoderm and subsequently vascular progenitor cells using the following differentiation protocol. Briefly, vascular smooth muscle cells were induced from iPS cells in a monolayer on ECM matrigel (Thermo Scientific) coated plates in ultraglutamine DMEM supplemented with 6 μM CHIR99021 and 100 μg/ml ascorbic acid. After 48 h of differentiation, CHIR99021 was retracted from the differentiation media and further differentiated until day 6. We performed a time course experiment to examine the expression of vascular smooth muscle cell markers (CD31 and CD34) during differentiation. Cells were analyzed at day 0, 2, 4 and 6 during VSMC differentiation.

Results

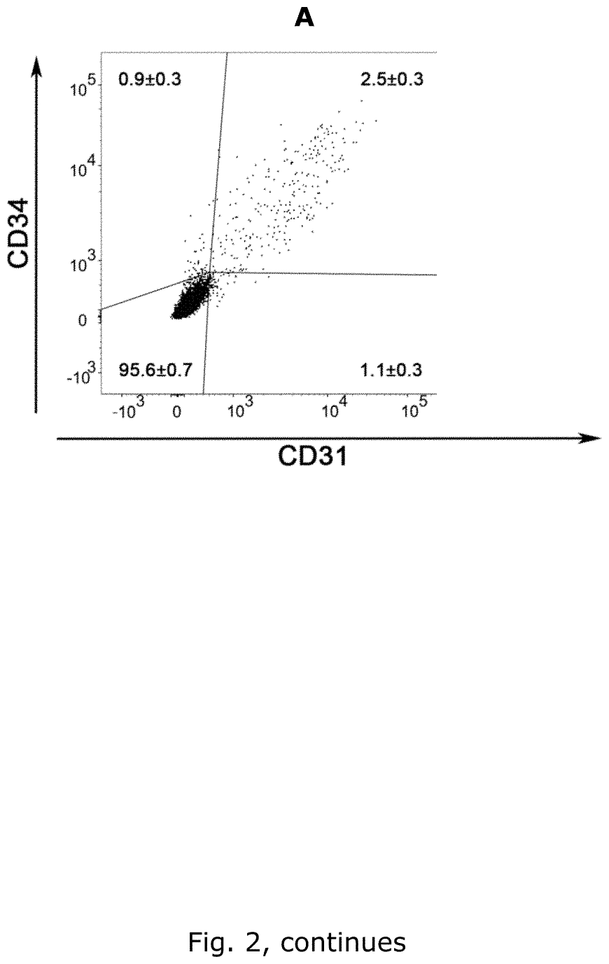

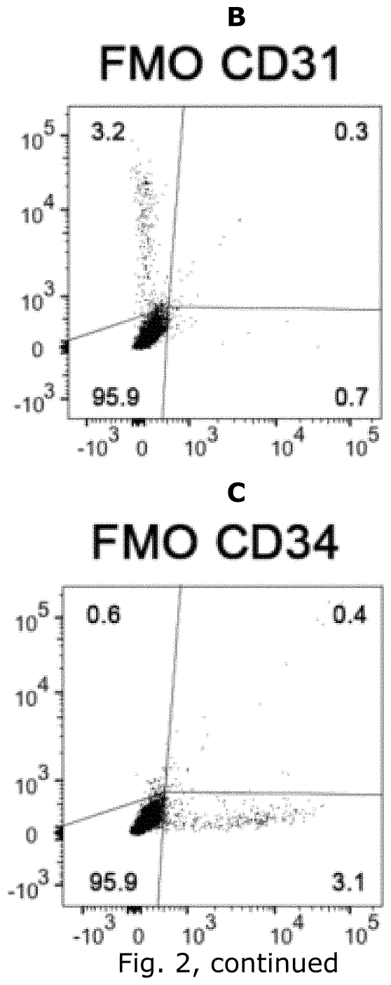

The endothelial and stem cell markers CD31 and CD34 increase in response to the vascular differentiation conditions, as shown in FIG. 2.

Conclusion

These data indicate that CD31+/CD34+ cells are important for vascular differentiation.

### Example 4

### Increasing the Amount of CD31+ Human ADRC's Improves Erection in Men With Erectile Dysfunction (ED) After Radical Prostatectomy

In this example, data from a phase 1 clinical trial examining the safety and potential effect of a single intracavernous injection of autologous ADRC' in patients with ED following radical prostatectomy (RP). The background and methods have previously been described in detail (Haahr et al.). Briefly, 21 men suffering from post RP ED, with no recovery using conventional therapy, were enrolled in a prospective phase 1 open-label as single arm study. All subjects had RP performed 5-18 months before enrollment, and were followed 12 months after intracavernosal transplantation. Erectile function was assessed by IIEF-5 scores. ADRC's were isolated using an automated processing Celution 800/CRS system following a liposuction from the patient himself under general anesthesia. ADRC's were characterized with respect to yield, colony forming activity and expression of CD31 and others analyzed by flow cytometry.

Results

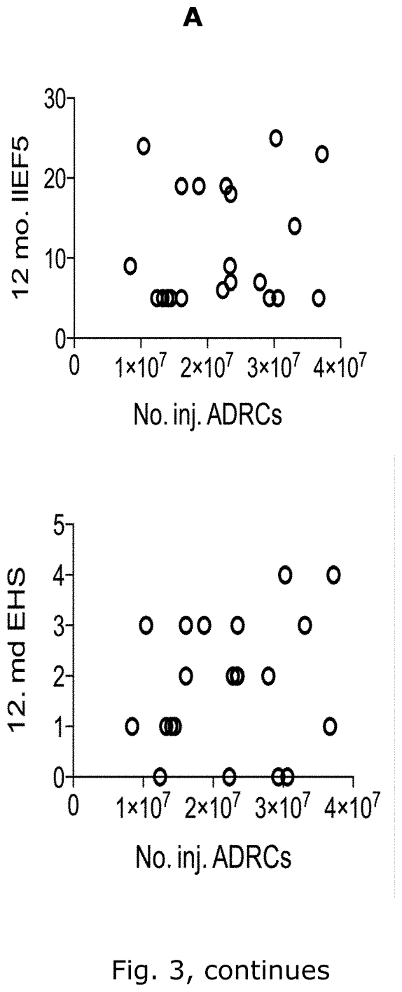

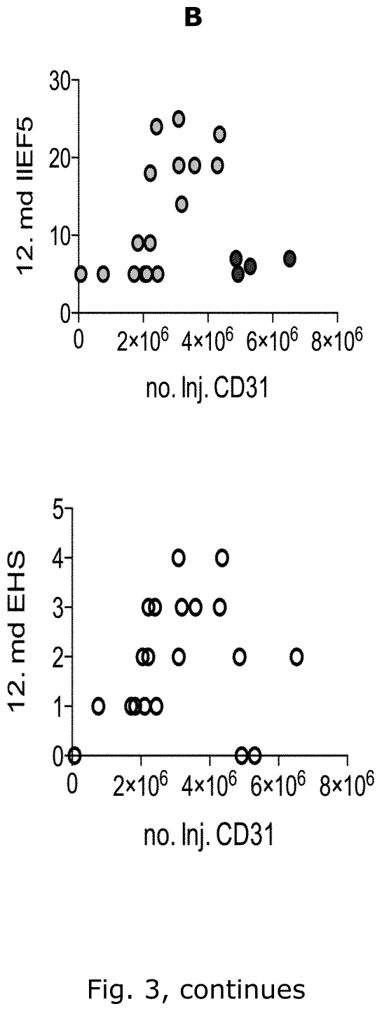

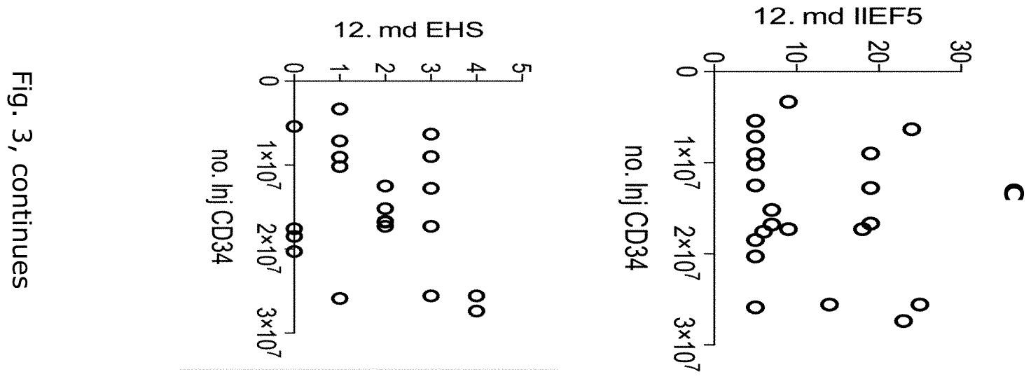

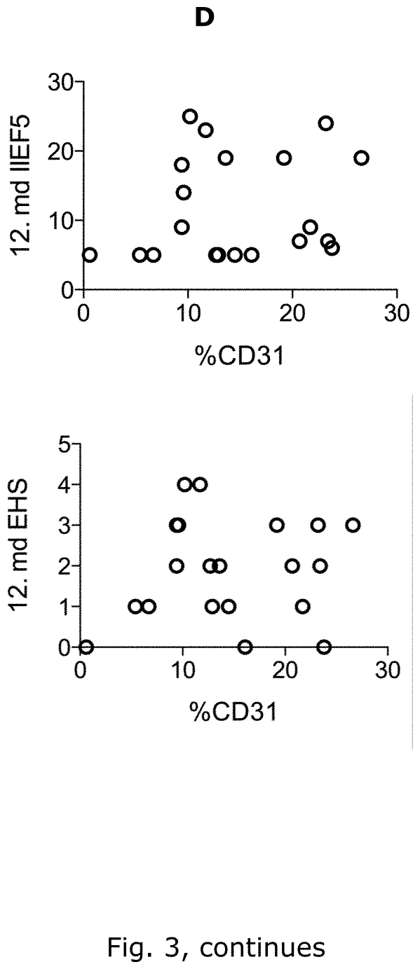

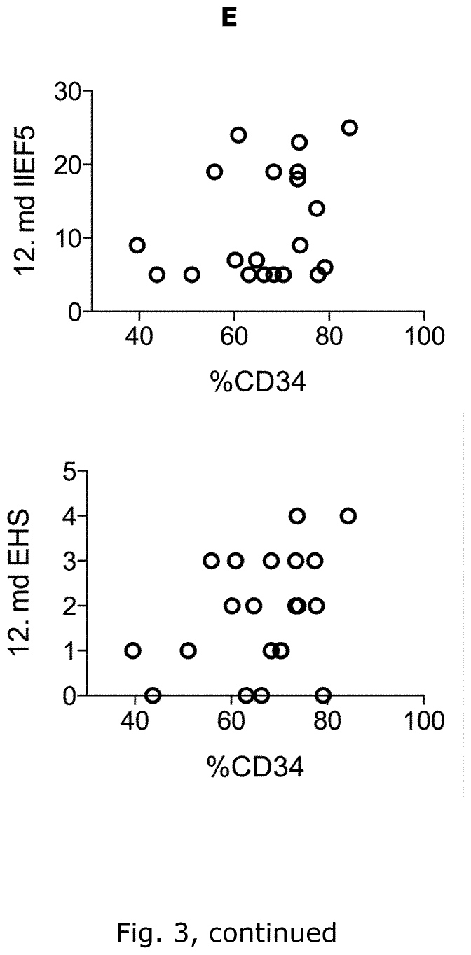

FIG. 3A shows that there is no apparent correlation on the effect on ED based on number of injected ADRC cells.

FIG. 3B shows that injection of increasing numbers from 0 to 4.2×10⁶ of CD31 expressing ADRC's strongly correlated with recovery of erectile function (FIG. 3, left, bright dots) when evaluated by the IIEF5 score. However, injection of more than 5×10⁶ cells had no effect (FIG. 3, left dark dots). A similar effect was seen when evaluated according to the EHS score. It is noted that according to the EHS score no upper negative effect of the number of CD31 cells could be seen. FIG. 3C shows that no such correlation was observed injecting increasing amounts of CD34. This was also seen for CD73 and CD90 (data not shown). FIG. 3D shows that at least 9% of the injected cells should be CD31+ to observe a clear positive effect according to both the IIEF5 and EHS scores. FIG. 3E shows that around at least 50% of the cells should be CD34 positive.

Conclusion

These data show that the recovery of erectile function is regulated by at least the amount of injected CD31 expressing cells in a dose-dependent manner. CD34+ positive cells appear also to be relevant.

### Example 5

### Increasing the Amounts of CD31+ human ADRC's reduces Breast Cancer Related Lymphedema

This example shows data from a phase 1 clinical trial examining the safety and potential effects of a single injection of autologous ADRC's in women with Breast Cancer Related Lymphedema (BCRL). The background and methods in this study have been described in detail previously (Toyserkani et al. Treatment of Breast Cancer-Related Lymphedema with Adipose-Derived Regenerative Cells and Fat Grafts: A Feasibility and Safety Study. Stem Cells Transl Med. 2017;6(8):1666-30 1672).

Materials and Methods

Briefly, ten women diagnosed with upper extremity lymphedema ISL stage I or II due to previous breast cancer treatment with lymph node involvement were included. All had been recurrence-free for minimum 1 year and the circumference of the affected arm was minimum 2 cm larger than the healthy arm.

ADRC's:

ADRC's were isolated from the patient's own adipose tissue using an automated processing Celution 800/IV system following liposuction under general anesthesia. ADRC's were characterized with respect to yield, colony forming activity and the 5 expression of CD31, CD34, CD235a and CD45 were analyzed by flow cytometry.

Assessment:

Lymphedema alleviation was evaluated by Patient-Reported Outcome

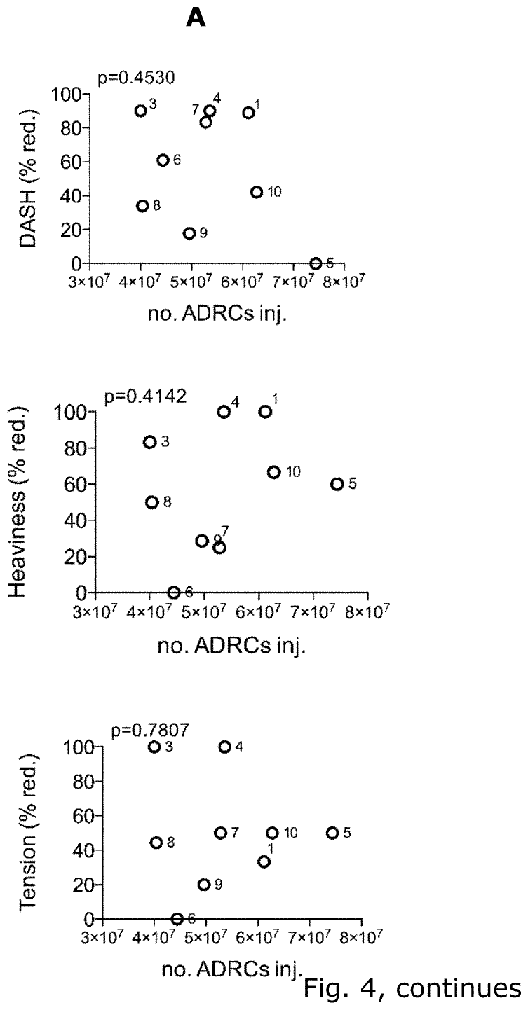

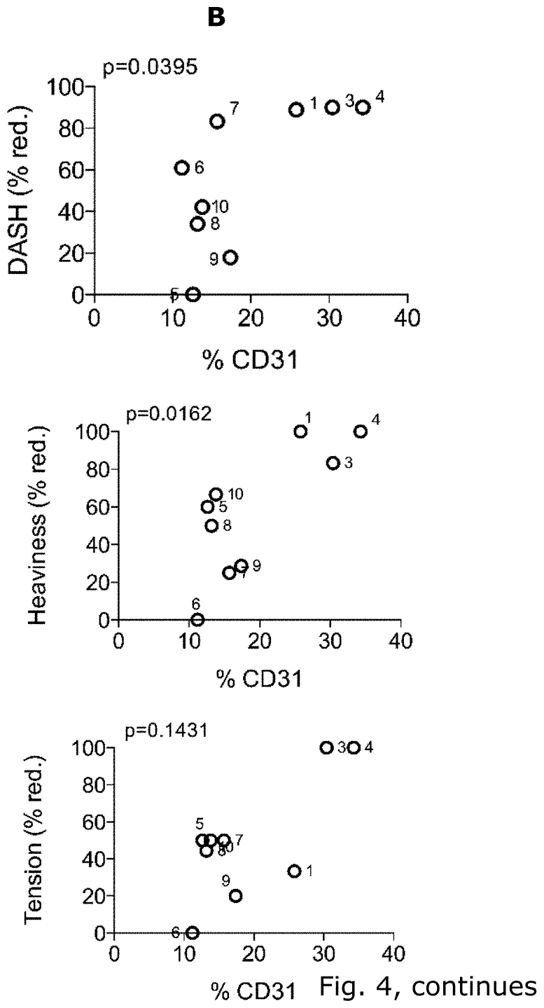

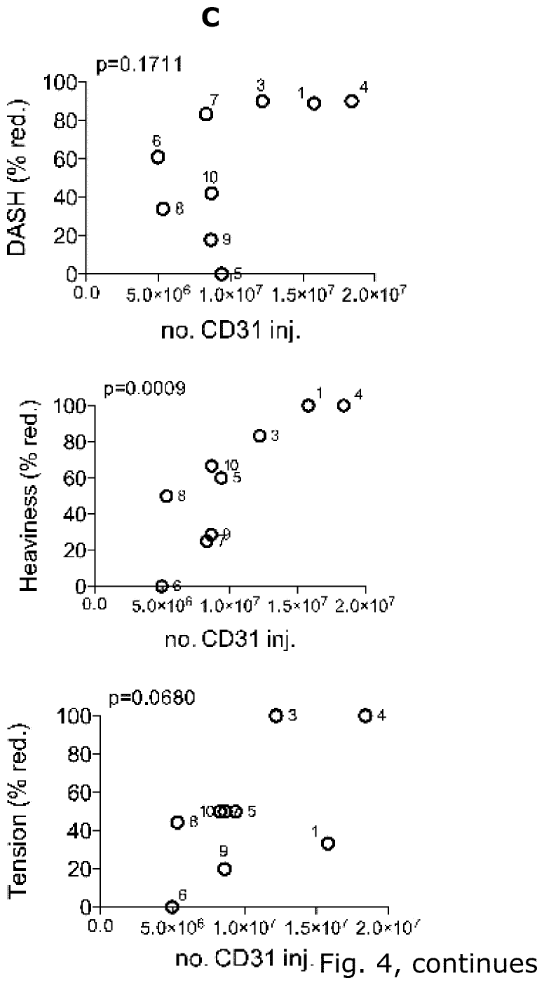

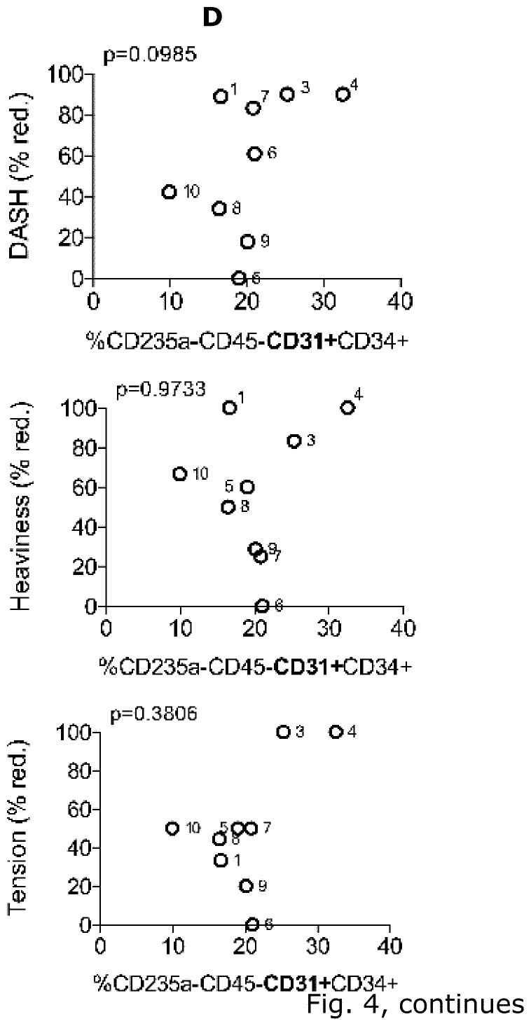

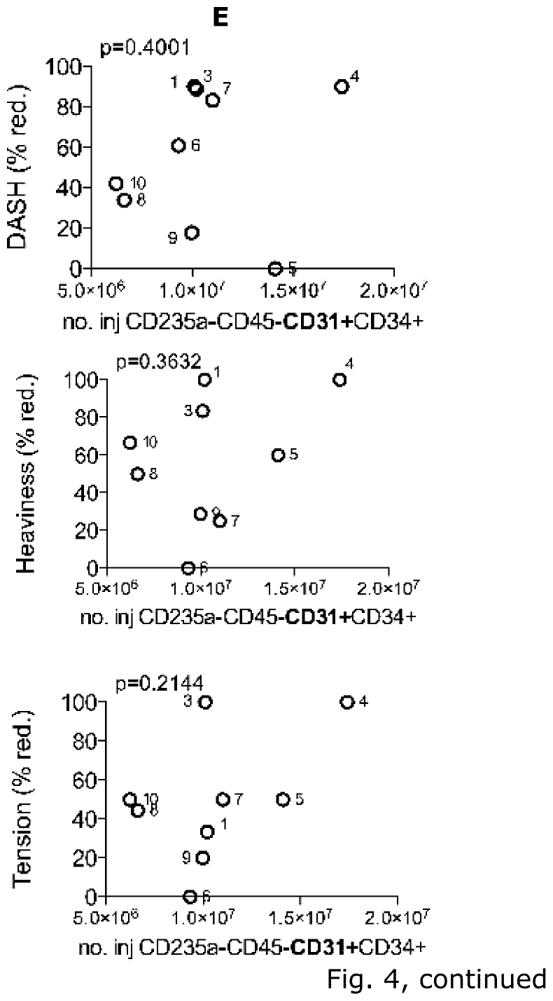

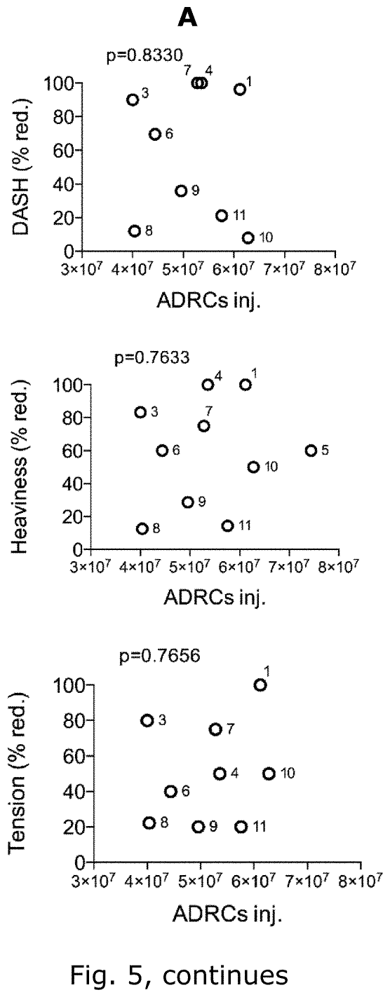

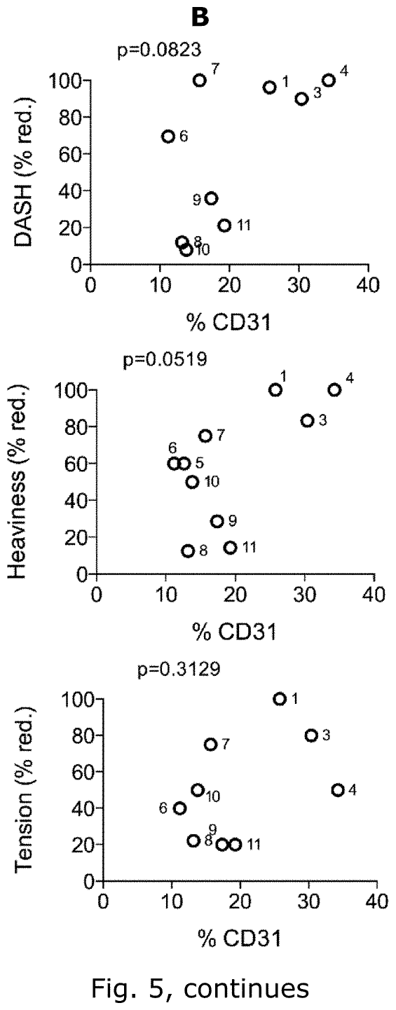

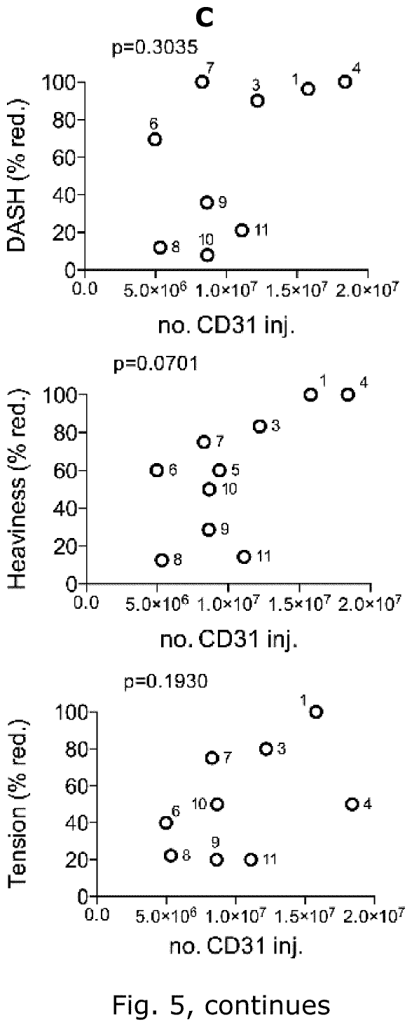

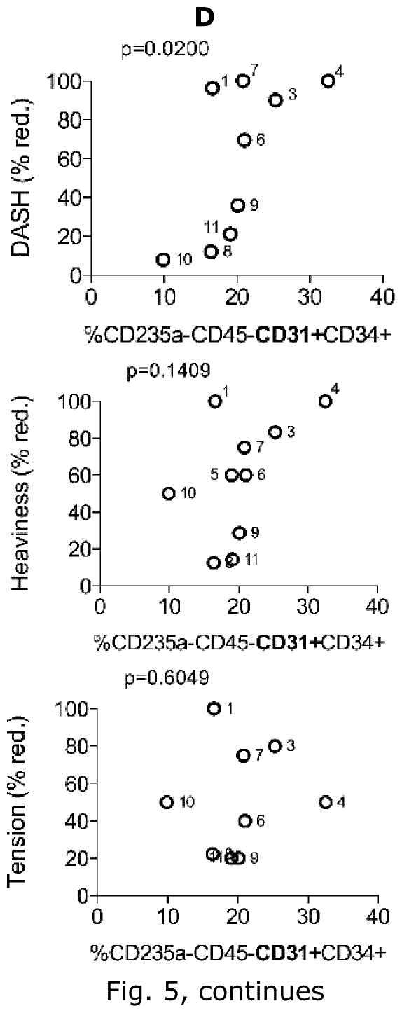

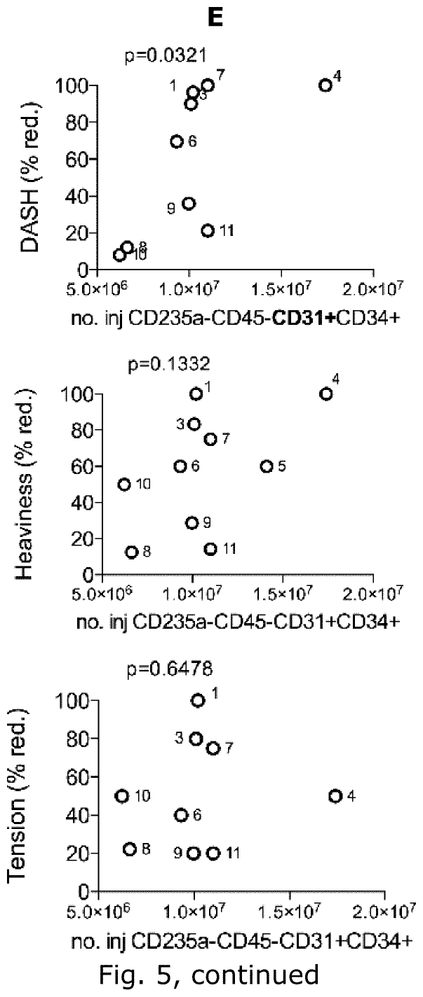

Assessment: Heaviness and tension in the lymphedema arm were both rated on a numerical rating scale ranging from 0 to 10, with 10 signifying the worst heaviness or tension imaginable. In addition, the Disabilities of the Arm, Shoulder and Hand (DASH) outcome questionnaire was used. Further, data was expressed as percentage reduction in scores from 0 to 6 months (((score, t=0—score, t=6 mo)/score, t=0)×100)). The higher the better (FIGS. 4 and 5).

Results

As seen in FIGS. 4 and 5, the data shows that injection of increasing numbers from 0 to 4.2×10⁶ of CD31 (CD31+) expressing ADRC's strongly correlated with recovery of lymphedema. Thus, compared to just looking at the number of injected ADRC's there is a clear relationship between injected CD31+ cells (both percentages and absolute numbers) and improvement in those patients.

Conclusion

These data clearly show that Lymphedema alleviation is regulated by the amount of injected CD31 expressing cells in a dose-dependent manner.

### Example 6

### Determination of CD31 Positive Populations

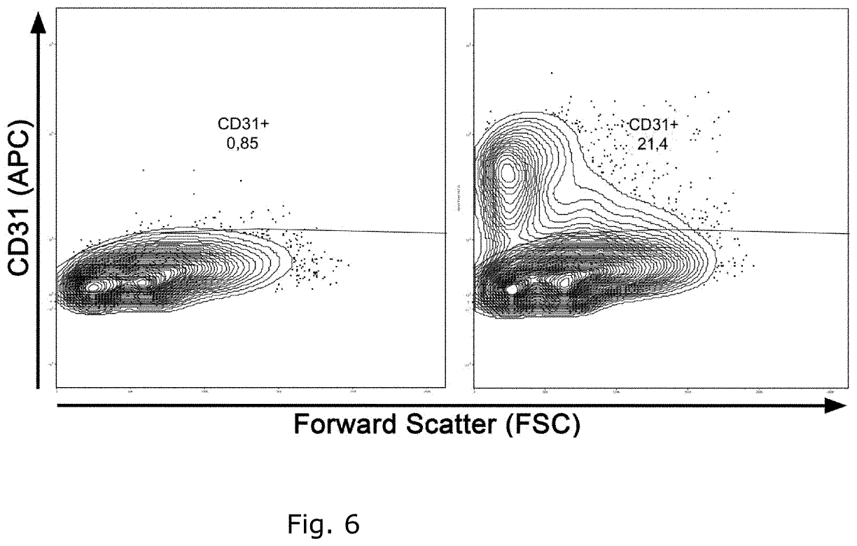

CD31 positive cells in the heterogeneous ADRC suspension were identified by flow cytometry and APC conjugated antibodies. An APC conjugated isotype control was used to mark the entire CD31 negative population (FIG. 6, left scatter plot) leaving less than 1% in the CD31+ gate. In this example the APC-CD31 stained sample (FIG. 6, right) showed that 20.55% (21.4−0.85) of the ADRCs were CD31+.

### Example 7

### ADRC-Derived CD31+ Cells Facilitate Tube Formation in Penile Tissue Ex Vivo

Aim of Example

To evaluate the ability of freshly isolated ADRCs, CD31-positive or CD31-negative cells to induce vascular tube formation.

Materials and Methods

Using corpus cavernous tissue explants from adult mice as an ex vivo angiogenesis model system, we compared the ability of freshly isolated, un-cultured human ADRCs, CD31-positive ADRCs or CD31-negative ADRCs to induce vascular tube formation in the penis-derived cells.

At day −1, corpus cavernous tissue explant cultures were established following dissection of mouse penile tissue. Corpus cavernous tissue was cut into smaller pieces and maintained in culture for 24 hours before further use. At day 0, human ADRCs were isolated from a lipoaspirate using the Cytori Celution IV device and a sample hereof further fractionated into CD31-positive and CD31-negative ADRCs by magnetic cell separation (human CD31 MicroBeads, Miltenyi Biotech). Immediately hereafter, cells from the original ADRC population or the CD31-positive and CD31-negative populations were then seeded at the same density in culture inserts (with a small pore size restricting migration of cells) and then co-cultured with the already established penile tissue explants for 7 days. For each experimental setup, VEGF (Vascular Endothelial Growth Factor—a potent stimulator for angiogenesis) and normal growth medium served as positive and negative controls (Ctrl) without cells in inserts, respectively. Phase contrast microscopic images of tubes sprouting from corpus cavernous tissue explant were converted to binary versions using ImageJ. The number of tubes emerging from 25 explants were quantified by the ImageJ plugin Tubeness. Values represent mean ± SEM of n=4 independent experiments; One-way ANOVA followed by a Tukeys multiple comparisons test was used to test the difference between the CD31-positive and CD31-negative group; *p=0.0126.

Results and Conclusion

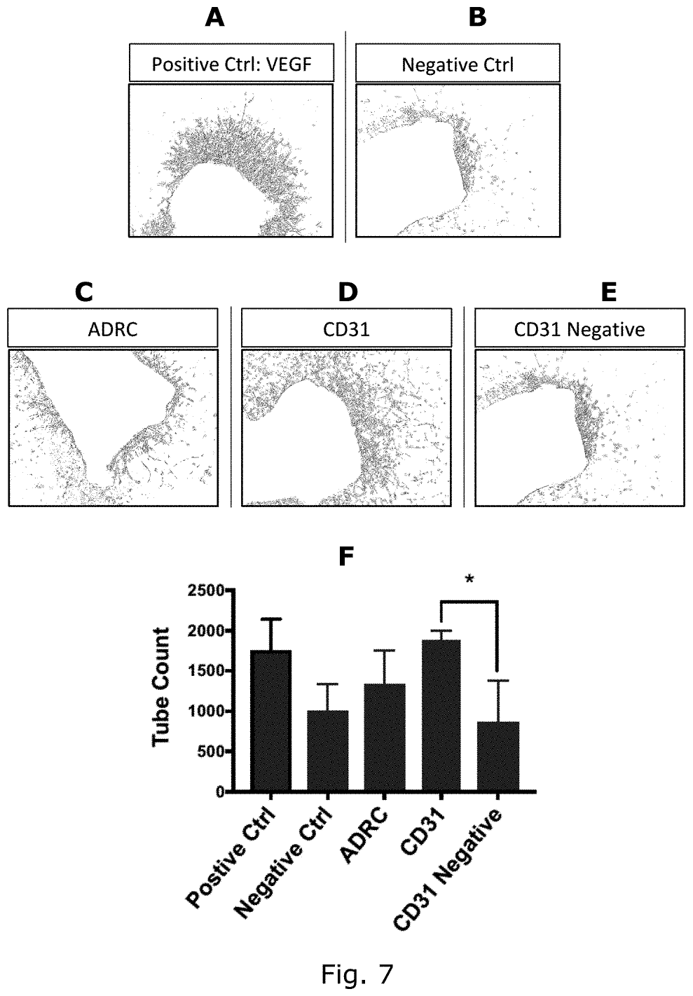

As seen in the representative images and summarized in FIG. 7 for all 4 experiments, the CD31-positive human ADRC subpopulation stimulates tube formation to a higher extent than the CD31-negative counterpart. Notably, the CD31-positive ADRCs seem to possess superior angiogenic properties compared to the unfractionated ADRCs. In contrast, the CD31-negative cells apparently have an inhibitory effect. These data strongly suggest that treatment with CD31-positive ADRCs will be superior to treatment with the original population due to enrichment of a more potent cell population in parallel with the elimination of an inhibitory population (the CD31-negative).

### Example 8

### Freshly Isolated Autologous Adipose Derived Regenerative Cells Effectively Heal Crohn's Disease Associated Fistulas

Aim of Study

Describes data from a phase 1 clinical trial examining the safety and potential effects of a single bolus of autologous ADRC's in patients with Crohn's complicated by perianal fistulas.

Materials and Methods

Briefly, 5 patients diagnosed with Crohn's disease and suffering from complications with perianal fistulas were included after MRI-scanning of the fistula. One patient was treated for 2 fistulas, whereas the remaining 4 patients were treated for one fistula.

ADRC's:

ADRC's were isolated from the patient's own adipose tissue using an automated processing Celution IV system following liposuction under general anesthesia. ADRC's were characterized with respect to yield (n=5) and the expression of CD31 was analyzed by flow cytometry (n=4). Cells were injected together with a small amount of adipose tissue around the fistula.

Assessment:

Fistula resolution was evaluated by clinical examination at 2 weeks, 12 weeks and 6 months for time to heal, recurrence as well as changes in Wexner score. A final MRI scanning was done at the 6 months control examination (3 of 5 patients to date).

Results

Patients received between 25-38 million ADRCs, with 17-56% identified by flow cytometry as being CD31+. This implies that patients were injected with 6.5-12.3 million CD31+ ADRCs. MRI and/or clinical examination revealed complete fistula resolution for 4 of the 5 patients (and 5 of 6 treated fistulas) with no recurrence in the study period. In the patient with two fistulas, one was closed while the other remained open. This coincides with this patient having the lowest percentage of CD31+ ADRCs (17%), the total no. of which was furthermore divided into two in order to treat both fistulas.

Conclusion

These data clearly show a profound capability of ADRCs to heal CD-associated perianal fistulas but also that efficacy may be regulated by the amount of injected CD31 expressing cells. In this case, 3.35 million appears to be a critical number, whereas ranges from 7.75 to 12.3 are therapeutically relevant.

1. A method for treatment and/or alleviation of a medical disorder in a subject in need thereof, comprising administering to the subject a composition comprising An isolated CD31⁺ cells derived from adipose-derived regenerative cells (ADRC) wherein the amount of CD31⁺ cells in the composition constitutes at least 5% of the adipose-derived regenerative cells (ADRC) and, wherein the medical disorder is selected from the group consisting of erectile dysfunction (ED), Morbus Crohn's disease and lymphedema. medicament.

2-31. (canceled)

32. The method according to claim 1, wherein the disorder is erectile dysfunction.

33. The method according to claim 1, wherein the disorder is erectile dysfunction following radical prostatectomy (RP).

34. The method according to claim 1, wherein the disorder is lymphedema.

35. The method according to claim 1, wherein the disorder is breast cancer related lymphedema.

36. The method according to claim 1, wherein the disorder is Morbus Crohn's disease.

37. The method according to claim 1, wherein, the CD31⁺ cells have been obtained from a subject within 24 hours before said use.

38. The method according to claim 1, wherein the CD31⁺ cells are non-expanded cells.

39. The method according to claim 1, wherein the composition is used as an autograft.

40. The method according to claim 1, wherein the isolated CD31⁺ cells are also CD34⁺.

41. The method according to claim 1, wherein said CD31⁺ cells are CD235a⁻ and/or CD45⁻.

42. The method according to claim 1, wherein the amount of CD31⁺ cells in the composition constitute at least 20% of the adipose-derived regenerative cells (ADRC).

43. The method according to claim 1, wherein the amount of CD31⁺ cells in the composition constitutes at least 80% of the adipose-derived regenerative cells (ADRC) cells.

44. The method according to claim 1, wherein at least 1.5×10⁶ of CD31⁺ cells are administered to the subject.

45. The method according to claim 1, wherein in the range 2×10⁶ to 5×10⁶ CD31⁺ cells are administered to the subject.

46. The method according to claim 1, wherein the amount of cells being CD34⁺ in the composition constitutes at least 50% of the adipose-derived regenerative cells (ADRC) cells.

47. A kit comprising:
i. a composition comprising isolated CD31⁺ cells derived from adipose-derived regenerative cells (ADRC), wherein the amount of CD31⁺ cells in the composition constitutes at least 5% of the adipose-derived regenerative cells (ADRC).; and
ii. instructions for the treatment and/or alleviation of a disorder, selected from the group consisting of erectile dysfunction (ED), lymphedema, and Morbus Crohn's disease.

48. A method for screening for the regenerative capacity of a population of adipose-derived regenerative cells (ADRC) from a subject, the method comprising:
having provided a population of isolated adipose-derived regenerative cells (ADRC) from a subject,
determining the number of (viable) cells in the population being CD31⁺;
comparing said number to a first reference level;
determining that:
said population of adipose-derived regenerative cells (ADRC) is indicative of a high regenerative capacity, if said number of CD31⁺ cells is above the first reference level, or
said population of adipose-derived regenerative cells (ADRC) is indicative of a low regenerative capacity, if said number of CD31⁺ cells is equal to or below said first reference level.
